(19) United States
(12) Patent Application Publication
(10) Pub. No.: US 2022/0062429 A1
(43) Pub. Date: Mar. 3, 2022

(54) METHOD FOR ENHANCING EXPRESSION OF MOISTURIZING-RELATED SUBSTANCE IN EPIDERMIS

(71) Applicant: J-Network, Inc., Huntington Beach, CA (US)

(72) Inventors: Tatsuro MIYOSHI, Huntington Beach, CA (US); Brian Charles KELLER, Huntington Beach, CA (US); Akira KODAMA, Huntington Beach, CA (US)

(21) Appl. No.: 17/398,121

(22) Filed: Aug. 10, 2021

(30) Foreign Application Priority Data
Aug. 31, 2020 (JP) 2020-146116

(51) Int. Cl. A61K 47/60; A61K 47/62; A61K 47/69

(52) U.S. Cl. CPC A61K 47/60; A61K 47/6903; A61K 47/62

(57) ABSTRACT

To enhance expression of a moisturizing-related substance in an epidermis by using a diacylglycerol PEG adduct, a method for enhancing expression of a moisturizing-related substance in an epidermis is provided which includes applying a diacylglycerol PEG adduct to the epidermis as an active ingredient. The moisturizing-related substance is filaggrin, profilaggrin, and/or caspase-14. The diacylglycerol PEG adduct is selected from a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GD012).

### CROSS-REFERENCE TO RELATED APPLICATIONS AND CLAIM TO PRIORITY

This application is related to application no. 2020-146116, filed Aug. 31, 2020 in Japan, the disclosure of which is incorporated herein by reference and to which priority is claimed.

### FIELD OF THE INVENTION

The present invention relates to a method for enhancing expression of a moisturizing-related substance in an epidermis.

### BACKGROUND OF THE INVENTION

Vesicles composed of a phospholipid and a surfactant are known and are also referred to as liposomes. Japanese Patent No. 4497765 presents a preparation method for causing spontaneous formation of vesicles by using a lipid mainly containing a diacylglycerol polyethylene glycol adduct (hereinafter, also called “diacylglycerol PEG adduct”) in place of a phospholipid and mixing it with water or a surfactant . Such vesicles are used in a drug delivery system that delivers a target substance, for example, a protein or an antibody, to cells in a living organism by encapsulating the substance in the vesicles or binding the substance to the surface of the vesicles .

A vesicle containing a diacylglycerol PEG adduct as a lipid has a form in which the surface thereof is covered by a hydrophilic PEG chain, and is excellent in permeability to a living organism and stability in blood. Japanese Patent No. 6297737 describes that a charged element is made to bind to the surface of vesicles containing a diacylglycerol PEG adduct to positively charge the vesicles, thereby improving permeability of the vesicles to a stratum corneum of an epidermis and retention of the vesicles in the stratum corneum.

Vesicles in a drug delivery system are recognized simply as carriers of a target substance. The vesicles are finally decomposed into individual molecules in a living organism, but the action of the molecules themselves constituting the vesicles in the living organism is not well known. U.S. Pat. Nos. 6,998,421 and 6,495,596 disclose some actions of a diacylglycerol PEG adduct in a living organism. According to the disclosures, the diacylglycerol PEG adduct binds to phospholipase A in the living organism to inhibit this enzyme, thereby exerting an anti-inflammatory action. However, the action of the diacylglycerol PEG adduct in the living organism is still unclear in many respects.

An object of the present invention is to take advantage of a newly found property of a diacylglycerol PEG adduct, in particular, to enhance expression of a moisturizing-related substance in an epidermis.

### SUMMARY OF THE INVENTION

In order to achieve the above object, the present invention provides the following constitution.

An aspect of the present invention provides a method for enhancing expression of a moisturizing-related substance in an epidermis, comprising applying a diacylglycerol PEG adduct to a human epidermis as an active ingredient, wherein the diacylglycerol PEG adduct is represented by

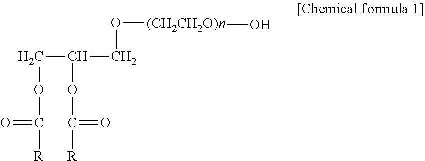

where the number of carbons in R in a long-chain fatty acid is in a range of 11 to 23 and n in a polyethylene glycol chain is in a range of 11 to 46.

Preferably, the diacylglycerol PEG adduct is selected from a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

Preferably, the diacylglycerol PEG adduct permeates an epidermis in a solution state or in a vesicle state.

Preferably, the moisturizing-related substance is filaggrin, profilaggrin, or caspase-14.

Preferably, the diacylglycerol PEG adduct is one of the ingredients of cosmetics or pharmaceuticals, and the method for enhancing expression of a moisturizing-related substance in an epidermis further comprises applying the cosmetics or pharmaceuticals on a surface of a human skin.

According to the present invention, a method for enhancing expression of a moisturizing-related substance in a human epidermis, using a diacylglycerol PEG adduct as an active ingredient is realized.

### BRIEF DESCRIPTION OF DRAWINGS

FIG. 1 illustrates test results of the filaggrin immunostaining method (in the upper portion) and the method of Hematoxylin Eosin staining (in the lower portion) for four samples;

FIG. 2a illustrates respective spot images for Samples 5, 6 and 7 related to GDM12;

FIG. 2b illustrates a production quantity of filaggrin for each sample determined based on FIG. 2a;

FIG. 3a illustrates respective spot images for Samples 8, 9 and 10 related to GDS23;

FIG. 3b illustrates a production quantity of filaggrin for each sample determined based on FIG. 3a;

FIG. 4a illustrates expression of profilaggrin mRNA for Samples 11, 12, and 13 related to GDS12;

FIG. 4b illustrates expression of profilaggrin mRNA for Samples 14, 15, and 16 related to GDM12;

FIG. 4c illustrates expression of profilaggrin mRNA for Samples 17 and 18 related to GDS23;

FIG. 5a illustrates expression of caspase-14 mRNA for Samples 19, 20, 21, 22, and 23 related to GDM12; and

FIG. 5b illustrates expression of caspase-14 mRNA for Samples 24 and 25 related to GDS23.

FIG. 6 is a graph which illustrates the measurement results of the water contents in the stratum corneum when the lotion is applied;

FIG. 7 is a graph which illustrates the measurement results of the average depth of total wrinkles when the lotion is applied;

FIG. 8 is a graph which illustrates the results of evaluation of the degree of wrinkles when the lotion is applied;

FIG. 9 is a graph which illustrates the measurement results of the water contents in the stratum corneum when the prescribed cream is applied;

FIG. 10 is a graph which illustrates the evaluation results of the degree of wrinkles when the prescribed cream is applied;

FIG. 11 is a graph which illustrates the measurement results of the area ratio of wrinkles when the prescribed cream is applied;

FIG. 12 is a graph which illustrates the measurement results of the water contents in the stratum corneum when the prescribed serum is applied;

FIG. 13 is a graph which illustrates the measurement results of the area ratio of wrinkles when the prescribed serum is applied;

FIG. 14 is a graph which illustrates the measurement results of the average depth of total wrinkles when the prescribed serum is applied;

### DETAILED DESCRIPTION OF THE PREFERRED EMBODIMENT(S)

Embodiments of the present invention are described below with reference to the drawings.

The present invention has been made by taking advantage of a newly found property of a diacylglycerol polyethylene glycol adduct (diacylglycerol PEG adduct). The newly found property is an action of enhancing expression of a moisturizing-related substance in a human epidermis.

A structural formula of a diacylglycerol PEG adduct, which is a lipid molecule according to the present invention, is schematically represented in Chemical formula 2.

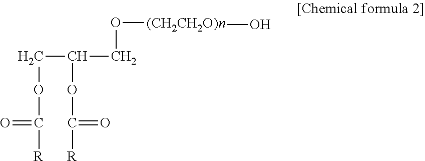

A diacylglycerol PEG adduct is composed of a glycerol skeletal part (CH₂CHCH₂) having three carbons, a PEG chain that is linear polyethylene glycol bonded to one terminal carbon of the three carbons in the skeletal part, and long-chain fatty acids (COOR) of the same type respectively bonded to the other two carbons of the three carbons. The PEG chain part is hydrophilic and the long-chain fatty acid parts are hydrophobic.

In the following description, when a specific diacylglycerol PEG adduct is described, it is referred to as “[PEG-n]+[glycerol]+[di]+[the name of long-chain fatty acid]” based on the type of long-chain fatty acid and n in the PEG chain. For example, when the long-chain fatty acid is myristic acid and n in the PEG chain is 12, the diacylglycerol PEG adduct is “PEG-12 glycerol dimyristate”. The specific diacylglycerol PEG adduct may also be abbreviated.

The number of carbons in R in long-chain fatty acid can be in a range of 11 to 23. Examples of long-chain fatty acids within this range include myristic acid, palmitic acid, stearic acid, and oleic acid. Further, n in the PEG chain can be in a range of 11 to 46. Examples of a diacylglycerol PEG adduct related to the present invention are as follows. The melting point and the abbreviation are described in parentheses.

PEG-12 glycerol dimyristate (25.0° C.: GDM12)

PEG-12 glycerol distearate (40.0° C.: GDS12)

PEG-23 glycerol distearate (39.8° C.: GDS23)

PEG-23 glycerol dipalmitate (31.2° C.: GDP23)

PEG-12 glycerol dioleate (25.0° C.: GDO12)

In a skin barrier function of a human epidermis, filaggrin in a lower portion of a stratum corneum is a protein having an important role. Filaggrin is first produced as profilaggrin in a stratum granulosum below the stratum corneum. Profilaggrin is decomposed by a plurality of enzymes into filaggrin. Filaggrin is further decomposed by a plurality of other enzymes such as caspase-14, into a natural moisturizing factor (NMF) in an upper portion of the stratum corneum. NMF has a buffering action of retaining moisture and maintaining pH in the stratum corneum. This action promotes normal differentiation of epidermal cells and reduces growth of pathogenic bacteria. It is said that expression of filaggrin is decreased in patients with atopic dermatitis.

In the present application, profilaggrin, filaggrin, and NMF that are moisturizing-related substances in an epidermis, and enzymes related to them, are collectively referred to as “moisturizing-related substances”.

The inventors have found that expression of filaggrin, profilaggrin, and caspase-14 that are moisturizing-related substances in an epidermis is enhanced by applying a diacylglycerol PEG adduct to a human epidermis. Enhancement of expression of these moisturizing-related substances indicates that NMF finally produced by these substances also increases. This action is a novel action of the diacylglycerol PEG adduct in the human epidermis, and is a novel property of the diacylglycerol PEG adduct. This property can provide a moisturizing effect to the epidermis. This effect is not merely a moisturizing effect that protects the surface of the epidermis from drying, but a moisturizing effect obtained by an action in a stratum corneum and an underlying stratum granulosum in the epidermis.

The present invention takes advantage of the newly found property of the diacylglycerol PEG adduct, thereby providing an expression enhancer for a moisturizing-related substance in an epidermis which contains the diacylglycerol PEG adduct as an active ingredient. Further, the present invention takes advantage of the newly found property of the diacylglycerol PEG adduct, thereby providing a method for enhancing expression of a moisturizing-related substance in an epidermis, the method including application of the diacylglycerol PEG adduct to the epidermis as an active ingredient.

In the present invention, when the diacylglycerol PEG adduct is applied to the human epidermis, the diacylglycerol PEG adduct may be used singly or in combination of two or more thereof.

According to the present invention, the diacylglycerol PEG adduct that has reached inside the epidermis can increase the production quantity of the moisturizing-related substance in the epidermis than in the absence of the diacylglycerol PEG adduct. As a result, not only the inside of the epidermis but also the surface of the epidermis is satisfactorily moisturized. Therefore, according to the present invention, it is possible to provide a moisturizer for the epidermis containing the diacylglycerol PEG adduct as an active ingredient, especially for cosmetics or pharmaceuticals. Further, according to the present invention, it is possible to provide a method for moisturizing the epidermis, which uses the diacylglycerol PEG adduct as an active ingredient. This method is expected to be effective especially as a therapeutic agent or a method of treatment for dermatosis such as atopic dermatitis or psoriasis. Cosmetics or pharmaceuticals containing the diacylglycerol PEG adduct as an active ingredient can be provided in various forms including, for example, an aqueous solution, emulsion, gel, and cream.

In one method for making the diacylglycerol PEG adduct reach inside the human epidermis, the diacylglycerol PEG adduct can be made to reach inside the epidermis in a solution state in which the diacylglycerol PEG adduct is dissolved in water or a predetermined solvent. For example, a diacylglycerol PEG adduct solution having a predetermined concentration is prepared using phosphate buffered saline (PBS(−)) as a solvent and the solution is applied to the skin surface, whereby the diacylglycerol PEG adduct can be made to permeate the epidermis. The applied solution permeates a stratum corneum as the uppermost layer, and further permeates a stratum granulosum below the stratum corneum. In each layer in the epidermis that the diacylglycerol PEG adduct permeates, the diacylglycerol PEG adduct enhances expression of a moisturizing-related substance originally present in that layer.

In another method for making the diacylglycerol PEG adduct reach inside the human epidermis, it is possible to make the diacylglycerol PEG adduct reach inside the epidermis in a vesicle state. Such vesicles are formed as closed spherical shells composed of a double layer of the diacylglycerol PEG adduct or a multilayer in which the double layers are stacked, and a hydrophilic PEG chain is arranged in the surface of the outermost layer. The vesicles of the diacylglycerol PEG adduct are prepared and applied on the skin surface, whereby the diacylglycerol PEG adduct can be made to permeate the epidermis. After reaching inside the epidermis, the vesicles are decomposed and separated into individual molecules, whereby the action of the diacylglycerol PEG adduct itself can be exerted.

In a conventional drug delivery system, a diacylglycerol PEG adduct as a material of vesicles has been considered as a mere carrier of a target substance. Meanwhile, the present invention uses a diacylglycerol PEG adduct itself as an active ingredient. Therefore, the present invention does not require a target substance to be incorporated into vesicles in a usual drug delivery system, basically. According to the present invention, vesicles formed by mixing water and the diacylglycerol PEG adduct only are made to permeate the epidermis, thereby making the diacylglycerol PEG adduct itself function as an expression enhancer for the moisturizing-related substance in the epidermis.

Some diacylglycerol PEG adducts spontaneously form vesicles by being mixed with water at a predetermined temperature (see Japanese Patent Nos. 4497765 and 6297737). For example, a suspension of GDM12 or GDO12 vesicles can be obtained by mixing and stirring 2 mass % of GDM12 or GDO12 in 98 mass % of deionized water at room temperature. In another example, a suspension of GDS12 or GDS23 vesicles can be obtained by dissolving 2 mass % of GDS12 or GDS23 at a temperature of 45° C. to 55° C. and then mixing and stirring it in 98 mass % of deionized water at a temperature of 45° C. to 55° C. In further another example, a suspension of GDP23 vesicles can be obtained by dissolving 2 mass % of GDP23 at 37° C. and then mixing and stirring it in 98 mass % of deionized water at 37° C. The vesicles are stable even when the suspension obtained at a temperature above room temperature is cooled to room temperature.

Also in a case of using vesicles formed by mixing and stirring the diacylglycerol PEG adduct and an aqueous solution of any of various substances in place of water as still another example, if this case is also included in the scope of the present invention, the substance contained in the aqueous solution can have another function.

The scope of the present invention also covers, as still another example, a case of modifying the surface of the vesicles formed by mixing and stirring water or the aqueous solution and the diacylglycerol PEG adduct with a charged element, for example, a cationic surfactant, and using such vesicles. Japanese Patent No. 6297737 describes that positively charged vesicles are excellent, in particular, in permeability to an epidermis and retention in the epidermis.

A relation between application of a diacylglycerol PEG adduct to an epidermis and a moisturizing-related substance in the epidermis is presented by test data below.

(1) Test 1 on Promotion of Filaggrin Production

After samples were prepared using a human epidermis model, production of filaggrin was observed by applying two staining methods, that is, an immunostaining method using an anti-filaggrin antibody and the method of Hematoxylin Eosin staining.

(1-1) Test Method

Treatment of Epidermis Model

A human three-dimensional cultured epidermis model (hereinafter, “epidermis model”) (LabCyteEPI-MODEL24 6D: from Japan Tissue Engineering Co., Ltd.) was subjected to treatment in the following manner to prepare samples.

First, the epidermis model was incubated in an agar medium at room temperature for 24 hours. Thereafter, the epidermis model was cultured in a medium (an assay medium: from Japan Tissue Engineering Co., Ltd.) at 37° C. for 48 hours.

Subsequently, medium exchange was performed, 30 μL of each of Samples 1 to 4 described below was applied on the surface of a stratum corneum of the epidermis model, and the culture was continued for 18 hours. Sample 1 as a control contains PBS(−) only. Samples 2 to 4 are solutions of GDM12 with different concentrations, using phosphate-buffered saline PBS(−) as a solvent.
Sample 1: Control for GDM12 (N.C.)
Sample 2: 1% solution of GDM12
Sample 3: 2% solution of GDM12
Sample 4: 3.5% solution of GDM12

Thereafter, the sample on the surface of the stratum corneum of the epidermis model was sucked up with a sterilized cotton swab, so that an excess portion of the sample was removed, then the epidermis model was continuously cultured without newly applying the sample to the surface of the stratum corneum, and the epidermis model was recovered 6 days after the application of each sample.

Preparation of Frozen Section of Epidermis Model and Filaggrin Immunostaining Method

The recovered epidermis model was embedded in a frozen tissue embedding agent (O.C.T. compound: from Sakura Finetek Japan Co., Ltd.) to prepare a frozen section. The frozen section was then fixed in cold acetone, immersed in PBS(−) containing 1% bovine serum albumin BSA, and blocked at room temperature for 1 hour.

Thereafter, an anti-filaggrin antibody (from GeneTex, Inc.) was made to react with that section at 37° C. overnight, and an anti-mouse IgG Alexa Fluor (registered trademark) 488 antibody (from Cell Signaling Technology, Inc.) was further made to react at 37° C. for 2 hours. Further, Hoechst (registered trademark) 33342 (from Invitrogen) was made to react at room temperature for 5minutes, thereby performing nuclear staining. Then, green fluorescence and blue fluorescence were observed using a fluorescence microscope. The green fluorescence indicates the presence of filaggrin. The blue fluorescence is for confirming that there is no abnormality in cells of the epidermis model.

Preparation of Frozen Section of Epidermis Model and Method of Hematoxylin Eosin Staining

The recovered epidermis model was embedded in a frozen tissue embedding agent (O.C.T. compound: from Sakura Finetek Japan Co., Ltd.) to prepare a frozen section. Thereafter, that section was hydrated with PBS(−), immersed in a hematoxylin solution to be stained, cleaned with running water, and then immersed in an eosin solution. That section was further cleaned with 70% ethanol, dehydrated with 95% ethanol, and then sealed. Thereafter, observation was performed. The method of Hematoxylin Eosin staining was performed for confirming that there was no abnormality in cells of the epidermis model.

(1-2) Test Results

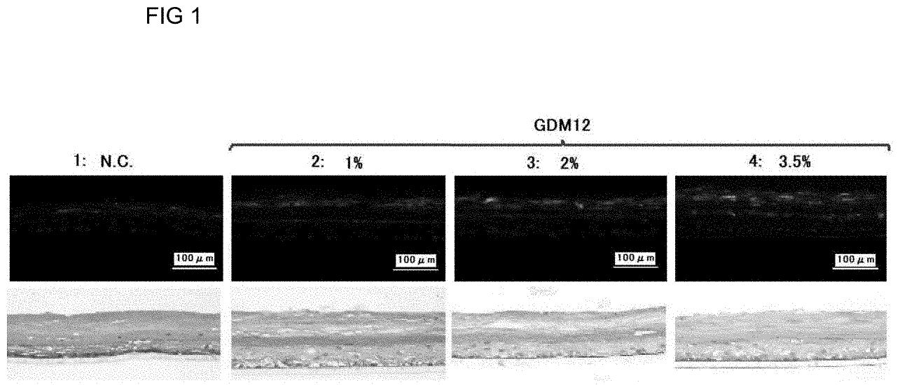

FIG. 1 illustrates test results of the filaggrin immunostaining method (in the upper portion) and the method of Hematoxylin Eosin staining (in the lower portion) for the four samples.

The green fluorescence (the upper white portion) in the upper fluorescence micrograph indicates that the production quantity of filaggrin in the stratum corneum of the epidermis model to which the GDM12 solution was applied is more than that of the epidermis model of N.C. Further, the production quantity of filaggrin is more as the concentration of the GDM12 solution is higher. Accordingly, an action of enhancing expression of filaggrin in an epidermis by GDM12 was confirmed.

The blue fluorescence (the center white portion) in the lowermost layer in the upper fluorescence micrograph and a lower image obtained by the method of Hematoxylin Eosin staining indicate that there is no abnormality in cells of the epidermis model.

(2) Test 2 on Promotion of Filaggrin Production

After samples were prepared using human epidermal cells, production of filaggrin was observed by dot blotting and the quantity of filaggrin was determined.

(2-1) Test Method

Treatment of Epidermal Cells

Normal human epidermal cells (hereinafter, “epidermal cells”) (from Kurabo Industries Ltd.) were seeded at a cell density of 1.0×10⁴ cells/well in a 96-well culture plate using a growth medium (KG2 medium: from Kurabo Industries Ltd.). After the cells were cultured at 37° C. under 5% carbon dioxide for 24 hours, the medium was replaced with media (KB2 media not containing bovine pituitary extract: from Kurabo Industries Ltd.) to which Samples 5 to 10 described below were respectively added. Each of Samples 5 and 8 as controls is a medium only. Samples 6, 7, 9, and 10 are different from one another in the type and/or concentration (mass % with respect to the medium) of a diacylglycerol PEG adduct to be added to the medium.
Sample 5: Control (N.C.)
Sample 6: GDM12 (0.0005%)
Sample 7: GDM12 (0.0010%)
Sample 8: Control (N.C.)
Sample 9: GDS23 (0.0025%)
Sample 10: GDS23 (0.0050%)

Thereafter, for each sample, the epidermal cells were cultured at 37° C. under 5% carbon dioxide for 72 hours. Subsequently, the epidermal cells were cleaned with PBS(−), 0.5% Triton X-100 (containing 2 mM PMSF) was then added, and thereafter the cells were disrupted by sonication.

Dot Blotting

A fixed amount of the suspension of disrupted cells was blotted on a nitrocellulose membrane and dried overnight. The transfer membrane after drying was immersed in a PBS solution of 1% BSA and blocked at room temperature for 1 hour. Thereafter, the membrane was cleaned with PBS(−), and an anti-human filaggrin antibody (Anti-Filaggrin: from ARGENE SA) was added onto the transfer membrane at a dilution concentration of 1:4000. The mixture was made to react at room temperature for 1 hour, and then the transfer membrane was cleaned with a PBS solution.

Thereafter, an immunohistochemical staining reagent (Histofine Simple Stain MAX-PO(M): from Nichirei Biosciences Inc.) was added onto the transfer membrane at a dilution concentration of 1:100 and was made to react at room temperature for 1 hour. After the transfer membrane was cleaned with PBS −), a chemiluminescent western blotting substrate (Lumi-Light Western Blotting Substrate: from Roche Diagnostics K.K.) was added onto the transfer membrane, and after 1 minute, a spot image of a chemiluminescent pattern was taken using a photographic device (Light-Capture: from ATTO CORPORATION).

Quantitative Determination Method for Filaggrin

The brightness of each spot in the spot image obtained by dot blotting was quantitively determined using an analyzer (CS Analyzer Version 2.0: from ATTO Corporation).

(2-2) Test Results

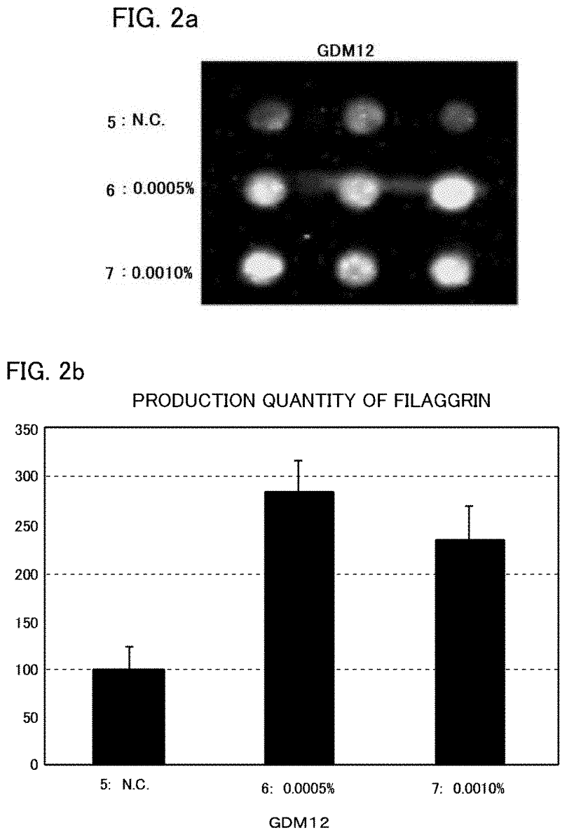

FIG. 2a illustrates respective spot images for Samples 5, 6 and 7 related to GDM12, and FIG. 2b illustrates the production quantity of filaggrin for each sample determined based on FIG. 2a. The vertical axis of the graph represents the relative quantity when the quantity for the control N.C. is assumed as 100% (the same representation is also applied to graphs in drawings that will be described later).

FIG. 2a shows that the production quantity of filaggrin in epidermal cells to which GDM12 was applied is more than that in epidermal cells of N.C. In the measured quantity graph in FIG. 2b, 200% or more of filaggrin production was confirmed for Samples 6 and 7 as compared to the production for Sample 5 (N.C.) . Accordingly, an action of enhancing expression of filaggrin in epidermal cells by GDM12 was confirmed.

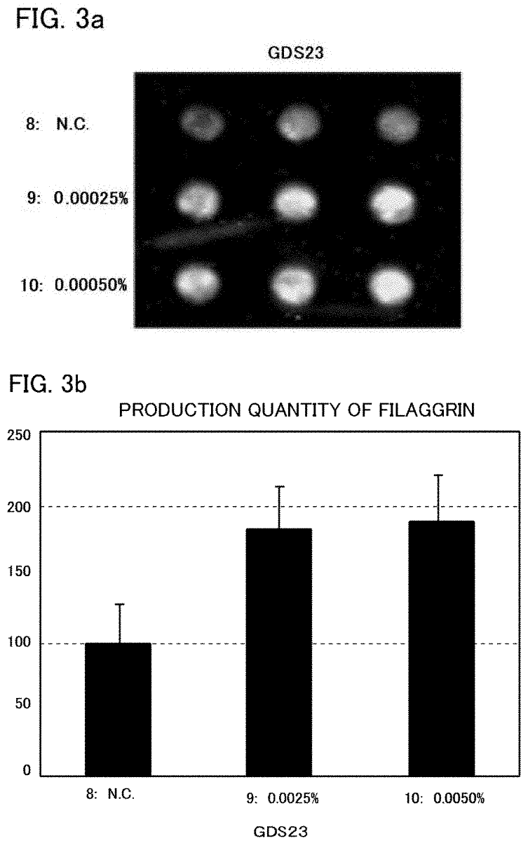

FIG. 3a illustrates respective spot images for Samples 8, 9 and 10 related to GDS23, and FIG. 3b illustrates the production quantity of filaggrin for each sample determined based on FIG. 3a.

FIG. 3a shows that the production quantity of filaggrin in epidermal cells to which GDS23 was applied is more than that in epidermal cells for N.C. In the measured quantity graph in FIG. 3b, about 200% of filaggrin production was confirmed for Samples 9 and 10 as compared with the production for Sample 8 (N.C.). Accordingly, an action of enhancing expression of filaggrin in epidermal cells by GDS23 was confirmed.

Enhancement of expression of filaggrin in a stratum corneum of an epidermis or in epidermal cells illustrated in FIGS. 1 to 3b shows that expression of NMF, which is a decomposition product of filaggrin, can also be enhanced.

(3) Amplification Test of Profilaggrin mRNA

After samples were prepared using human epidermal cells, RNA of the cells was extracted and the quantity of mRNA of profilaggrin was determined.

(3-1) Test Method

Treatment of Epidermal Cells

Normal human epidermal cells (hereinafter, “epidermal cells”) (from Kurabo Industries Ltd.) were seeded at a cell density of 2.0×10⁴ cells/well in a 96-well culture plate using a growth medium (KG2 medium: from Kurabo Industries Ltd.). After the cells were cultured at 37° C. under 5% carbon dioxide for 24 hours, the media were replaced with media (KB2 media not containing bovine pituitary extract: from Kurabo Industries Ltd.) to which Samples 11 to 18 described below were respectively added. Each of Samples 11, 14, and 17 as controls is a medium only. Samples 12, 13, 15, 16, and 18 are different from one another in the type and/or concentration (mass % with respect to the medium) of a diacylglycerol PEG adduct.
Sample 11: Control (N.C.)
Sample 12: GDS12 (0.100%)
Sample 13: GDS12 (0.050%)
Sample 14: Control (N.C.)
Sample 15: GDM12 (0.001%)
Sample 16: GDM12 (0.004%)
Sample 17: Control (N.C.)
Sample 18: GDS23 (0.002%)

Thereafter, the epidermal cells were cultured at 37° C. under 5% carbon dioxide for 3 hours. Subsequently, RNA was extracted from the cells.

Method for Analyzing Expression of Profilaggrin mRNA

Reverse transcription of the extracted RNA was performed to generate cDNA, and the quantity of profilaggrin mRNA was determined by quantitative real-time PCR expression analysis. Cyclophilin was used as an internal standard. In the analysis, the expression level of profilaggrin was corrected with a value of the expression level of cyclophilin as the internal standard in the same sample, and thereafter the correction value for each sample was calculated assuming the correction value for the control N.C. as 100%.

(3-2) Test Results

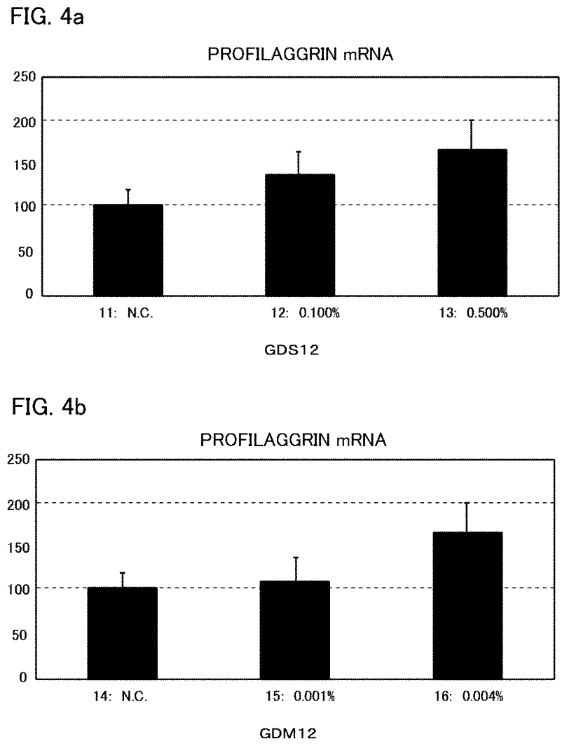

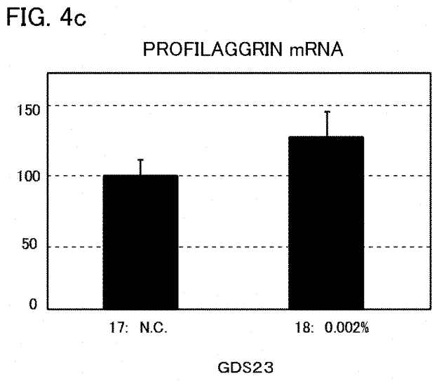

FIG. 4a illustrates expression of profilaggrin mRNA for Samples 11, 12, and 13 related to GDS12. FIG. 4a shows that expression of profilaggrin mRNA in epidermal cells to which GDS12 of Sample 12 or 13 was applied has been increased to about 150% as compared with that in epidermal cells of Sample 11 as N.C. Further, for Samples 12 and 13, the increase is larger as the concentration of GDS12 is higher. Accordingly, an action of enhancing expression of profilaggrin in epidermal cells by GDS12 was confirmed.

FIG. 4b illustrates expression of profilaggrin mRNA for Samples 14, 15, and 16 related to GDM12. FIG. 4b shows that expression of profilaggrin mRNA in epidermal cells to which GDM12 of Sample 15 or 16 was applied has been increased as compared with that in epidermal cells of Sample 14 as N.C. Although the increase is slight for Sample 15 having a lower concentration of GDM12, the increase is about 150% for Sample 16 having a higher concentration of GDM12. Accordingly, an action of enhancing expression of profilaggrin in epidermal cells by GDM12 was confirmed.

FIG. 4c illustrates expression of profilaggrin mRNA for Samples 17 and 18 related to GDS23. FIG. 4c shows that expression of profilaggrin mRNA in epidermal cells to which GDS23 of Sample 18 was applied has been increased to about 120% as compared with that in epidermal cells for Sample 17 as N.C. Accordingly, an action of enhancing expression of profilaggrin in epidermal cells by GDS23 was confirmed.

Enhancement of expression of profilaggrin in epidermal cells illustrated in FIGS. 4a to 4c shows that expression of filaggrin as a decomposition product of profilaggrin and expression of NMF as a decomposition product of filaggrin can also be enhanced.

(4) Caspase-14 Amplification

After samples were prepared using human epidermal cells, RNA of the cells was extracted and the quantity of caspase-14 mRNA was determined.

(4-1) Test Method

Treatment of Epidermal Cells

Normal human epidermal cells (hereinafter, “epidermal cells”) (from Kurabo Industries Ltd.) were seeded at a cell density of 2.0×10⁴ cells/well in a 96-well culture plate using a growth medium (KG2 medium: from Kurabo Industries Ltd.). After the cells were cultured at 37° C. under 5% carbon dioxide for 24 hours, the media were replaced with media (KB2 media not containing bovine pituitary extract: from Kurabo Industries Ltd.) to which Samples 19 to 26 described below were respectively added. Each of Samples 19 and 24 as controls is a medium only. Samples 20, 21, 22, 23, and 25 are different from one another in the type and/or concentration (mass % with respect to the medium) of a diacylglycerol PEG adduct contained in the medium.
Sample 19: Control (N.C.)
Sample 20: GDM12 (0.001%)
Sample 21: GDM12 (0.004%)
Sample 22: GDM12 (0.020%)
Sample 23: GDM12 (0.100%)
Sample 24: Control (N.C.)
Sample 25: GDS23 (0.0004%)

Subsequently, the epidermal cells were cultured at 37° C. under 5% carbon dioxide for 3 hours. Subsequently, RNA was extracted from the cells.

Analysis of Expression of Caspase-14 mRNA

Reverse transcription of the extracted RNA was performed to generate cDNA, and the quantity of caspase-14 mRNA was determined by quantitative real-time PCR expression analysis. Cyclophilin was used as an internal standard. In the analysis, the expression level of caspase-14 was corrected with a value of the expression level of cyclophilin as the internal standard in the same sample, and thereafter the correction value of each sample was calculated, assuming the correction value of the control sample as 100%.

(4-2) Test Results

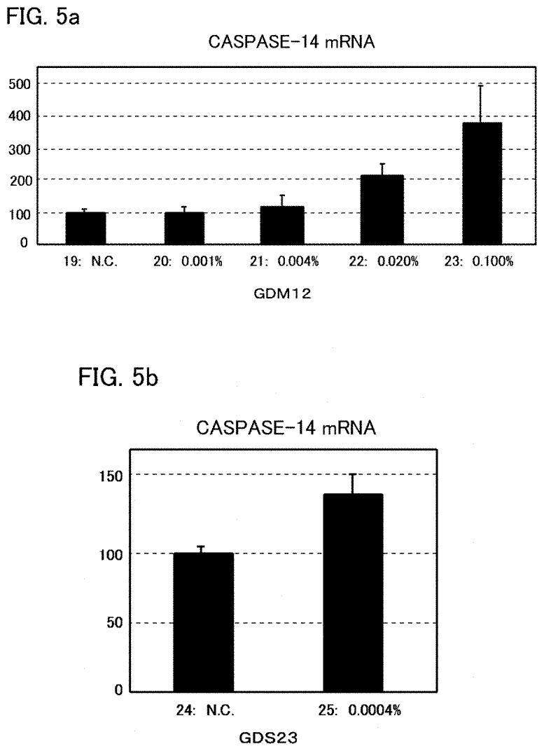

FIG. 5a illustrates expression of caspase-14 mRNA for Samples 19, 20, 21, 22, and 23 related to GDM12. FIG. 5a shows that expression of caspase-14 mRNA in epidermal cells to which GDM12 of each of Samples 20, 21, 22, and 23 was applied has been increased as compared with that in epidermal cells for Sample 19 as N.C. In addition, the increase is larger as the concentration of GDM12 is higher, and expression is increased to about 200% for Sample 22 and to about 400% for Sample 23. Accordingly, an action of enhancing expression of caspase-14 in epidermal cells by GDM12 was confirmed.

FIG. 5b illustrates expression of caspase-14 mRNA for Samples 24 and 25 related to GDS23. FIG. 5b shows that expression of caspase-14 mRNA in epidermal cells to which GDS23 of Sample 25 was applied has been increased to about 150% as compared with that in epidermal cells for Sample 24 as N.C. Accordingly, an action of enhancing expression of caspase-14 in epidermal cells by GDS23 was confirmed.

Enhancement of expression of caspase-14 in epidermal cells illustrated in FIGS. 5a and 5b shows that one of enzymes decomposing filaggrin into NMF is increased, and therefore shows that NMF can also be enhanced as a result.

(5) Test on Improvement in Fine Lines/Wrinkles by Lotion.

A test was conducted on how well wrinkles of a human face caused by dryness would improve by applying a lotion including diacylglycerol PEG. The component composition of the prepared lotion (wt %) is as follows:
WATER: 78.80
PROPANEDIOL: 15
BUTYLENE GLYCOL: 7
PEG-12 GLYCERYL DIMYRISTATE: 1.5
PEG-23 GLYCERYL DISTEARATE: 1.5
SQUALANE: 1.2
STEARAMIDOPROPYL DIMETHYLAMINE: 0.4
LACTIC ACID: 0.25
CHOLESTEROL: 0.15
PHENOXYETHANOL: 0.2

(5-1) Test Method

The Water Content in the Stratum Corneum

The test was conducted under the following conditions:
Each of the subjects washed her face twice by a prescribed face cleansing cream and a prescribed facial wash, and leaving it (letting the face naturalized) for more than 20 minutes in the environmental test room (room temperature: 21±1 ° C., humidity: 50±5%).
The water content in the stratum corneum was measured on the part on which the lotion was applied, and also on the part on which the lotion was not applied.
The test was conducted twice:
1st time: On the first day (day 0) before the subjects started to use the lotion.
2nd time: After the subjects used the lotion twice a day (morning and night (after the bath)) every day for the four weeks.
Measurement was made on the same parts (of the applied and unapplied parts) five times. The average value of the measurement results, from which the highest value and the lowest value were eliminated first, was adopted.

Average Depth of the Maximum (Total) Wrinkles

The test was conducted under the condition described as follows:
Each of the subjects washed her face twice by a prescribed face cleansing cream and a prescribed facial wash, and leaving it (letting the face naturalized) for more than 20 minutes in the environmental test room (room temperature: 21±1° C., humidity: 50±5%).
Replica was taken from the part on which the lotion was applied, and also on the part on which the lotion was not applied on the day 0 and after four weeks as described below:
The test was conducted twice:
1st time: on the first day (day 0) before the subjects started to use the lotion.
2nd time: After the subjects used the lotion twice a day (morning and night (after the bath)) every day for four weeks.

By using the image analysis system, the depth of wrinkles was measured from the replica taken.

Evaluation of the Degree of Wrinkles

The test was conducted under the following conditions:
Each of the subjects washed her face twice by a prescribed face cleansing cream and a prescribed facial wash, and leaving it (letting the face naturalized) for more than 20 minutes in the environmental test room (room temperature: 21±1° C., humidity: 50±5%).
Photos were taken on the part on which the lotion was applied, and also on the part on which the lotion was not applied on the day 0 and after four weeks as described below:
The test was conducted twice:
1st time: On the first day (day 0) before the subjects started to use the lotion.
2nd time: After the subjects used the lotion twice a day (morning and night (after the bath)) every day for the four weeks.
Judgment by visual inspection was made on the photos taken by an evaluator of the degree of wrinkles.

(5-2) Test Results

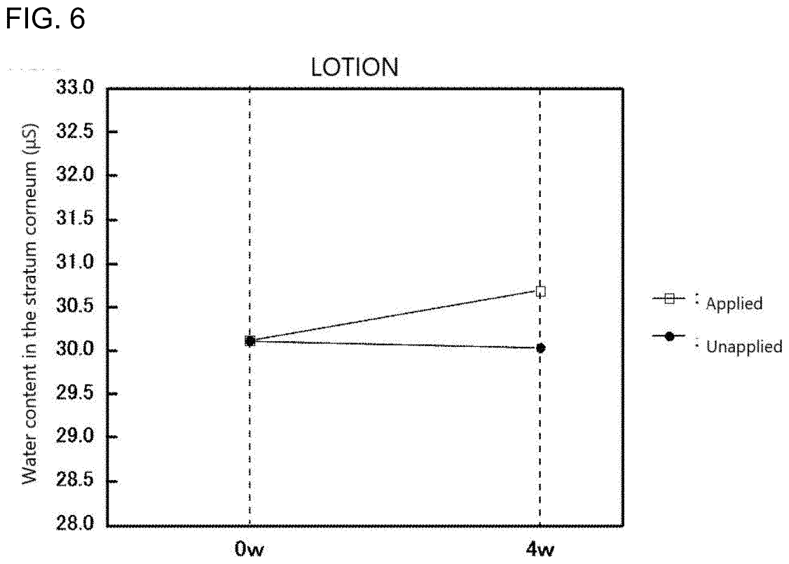

FIG. 6 shows the measurement results of the water content in the stratum corneum when the lotion is applied.

At the applied dermal site, a significant rise in the amount of the water content in the stratum corneum was recognized from the day 0 (30.086±0.159) to four weeks later (30.709±0.425).

On the other hand, at the unapplied dermal site, the amount of the water content in the stratum corneum was decreased from the day 0 (30.102±0.137) to the four weeks later (30.027±0.128).

Also, a significant increase was seen in the amount of change in the applied dermal site, compared with the amount of change in the unapplied dermal site.

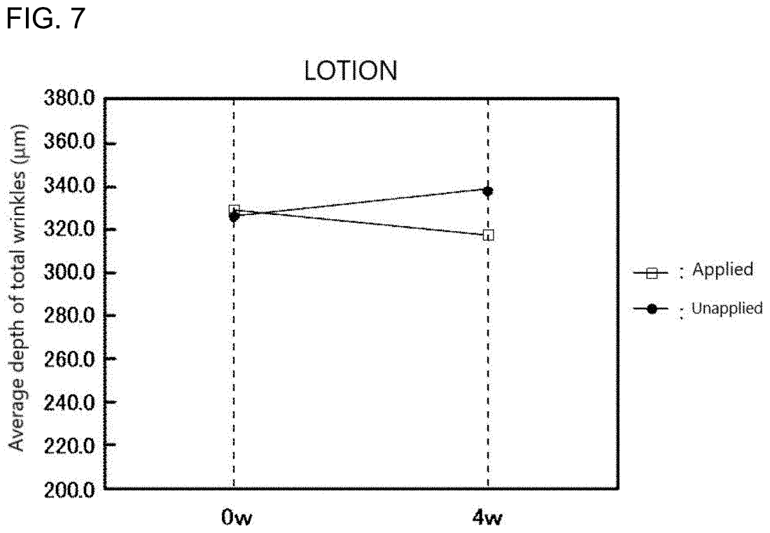

FIG. 7 is a graph which illustrates the measurement results of the average depth of the maximum wrinkles when the lotion is applied.

No significant difference was recognized in between the results on the applied dermal site and that of the unapplied dermal site on the initial day (day 0) and after the four weeks.

On the one hand, as for the amount of change (−6.107±13.462) in the applied dermal site, a significant decline was found compared to the amount of change (5.529±13.400) in the unapplied dermal site.

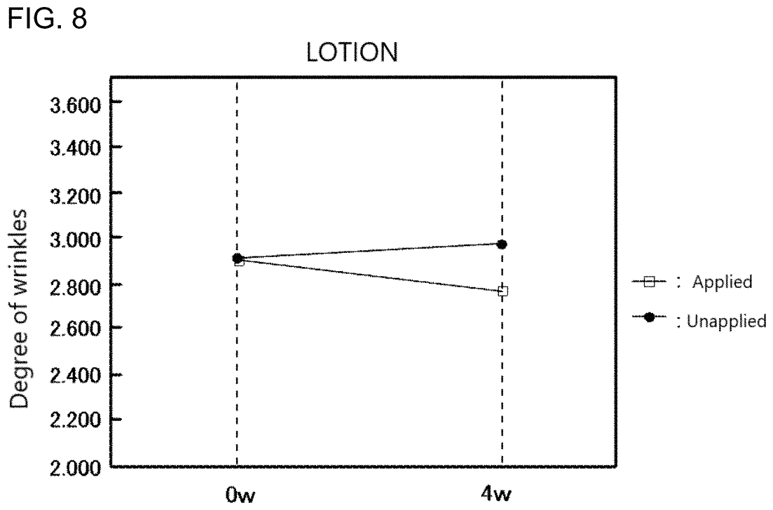

FIG. 8 is a graph which illustrates the evaluation results of degree of wrinkles when the lotion is applied.

At the applied dermal site, a significant decline was recognized from the day 0 (2.900±0.311) to the four weeks later (2.767±0.291).

Also, a significant decline was seen in the amount of change (−0.133±0.186) in the applied dermal site, compared to the amount of change (0.050±0.104) in the unapplied dermal site.

(6) Test on the Improvement in Fine Lines/Wrinkles by Prescribed Cream

A test was conducted on how well wrinkles of a human face caused by dryness would improve by the prescribed cream including diacylglycerol PEG.

The component composition of the prepared cream (wt %) is as follows:
WATER: 52.45
SQUALANE: 20
GLYCERIN: 8
BUTYLENE GLYCOL: 6
CETYL ALCOHOL: 4
PEG-23 GLYCERYL DISTEARATE: 2
PEG-12 GLYCERYL DIMYRISTATE: 2
GLYCERYL STEARATE: 2
PEG-45 STEARATE: 1.6
CHOLESTERYL MACADAMIATE: 1
CHOLESTEROL: 0.5
SODIUM CITRATE: 0.1
CITRIC ACID: 0.05
PHENOXYETHANOL: 0.3

(6-1) Test Method

The Water Content in the Stratum Corneum

The test was conducted under the condition described as follows:
Each of the subjects washed her face twice by a prescribed face cleansing cream and a prescribed facial wash, and leaving it (letting the face naturalized) for more than 20 minutes in the environmental test room (room temperature: 21±1° C., humidity: 50±5%).
The water content in the stratum corneum was measured on the part on which the cream was applied, and also on the part on which the cream was not applied.
The test was conducted twice:
1st time: On the first day (day 0) before the subjects started to use the cream.
2nd time: After the subjects used the cream twice a day (morning and night (after the bath)) every day for the four weeks.
Measurement was made on the same parts (of the applied and unapplied parts) five times. The average value of the measurement results, from which the highest value and the lowest value were eliminated first, was adopted.

Evaluation of the Degree of Wrinkles

The test was conducted under the following conditions:
Each of the subjects washed her face twice by a prescribed face cleansing cream and a prescribed facial wash, and leaving it (letting the face naturalized) for more than 20 minutes in the environmental test room (room temperature: 21±1° C., humidity: 50±5%).
Photos were taken on the part on which the cream was applied, and also on the part on which the cream was not applied.
The test was conducted twice:
1st time: On the first day (day 0) before the subjects started to use the cream.
2nd time: After the subjects used the cream twice a day (morning and night (after the bath)) every day for the four weeks.
Judgment by visual inspection was made on the photos taken by an evaluator of the degree of wrinkles.

Area Ratio of Wrinkles

The test was conducted under the condition described as follows:
Each of the subjects washed her face twice by a prescribed face cleansing cream and a prescribed facial wash, and leaving it (letting the face naturalized) for more than 20 minutes in the environmental test room (room temperature: 21±1° C., humidity: 50±5%).
Replica was taken from the part on which the cream was applied, and also on the part on which the cream was not applied.
The test was conducted twice:
1st time: on the first day (day 0) before the subjects started to use the cream.
2nd time: After the subjects used the cream twice a day (morning and night (after the bath)) every day for four weeks.

By using the image analysis system, the area ratio of wrinkles was measured from the replica taken.

(6-2) Test Results

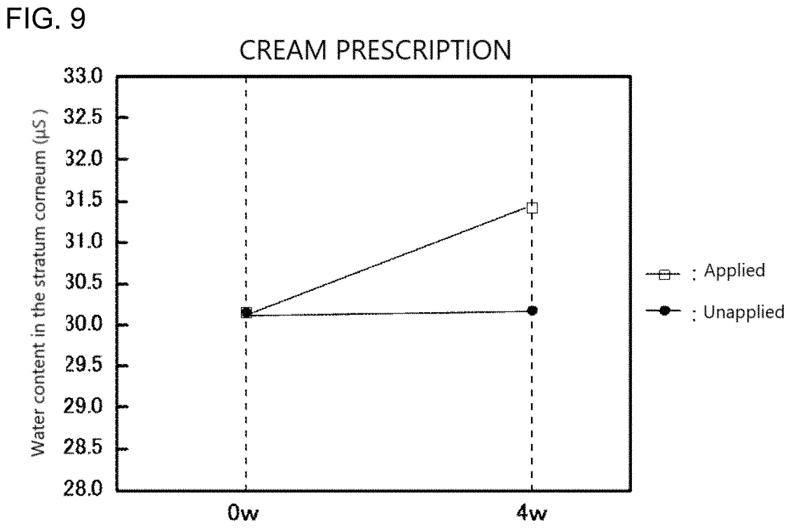

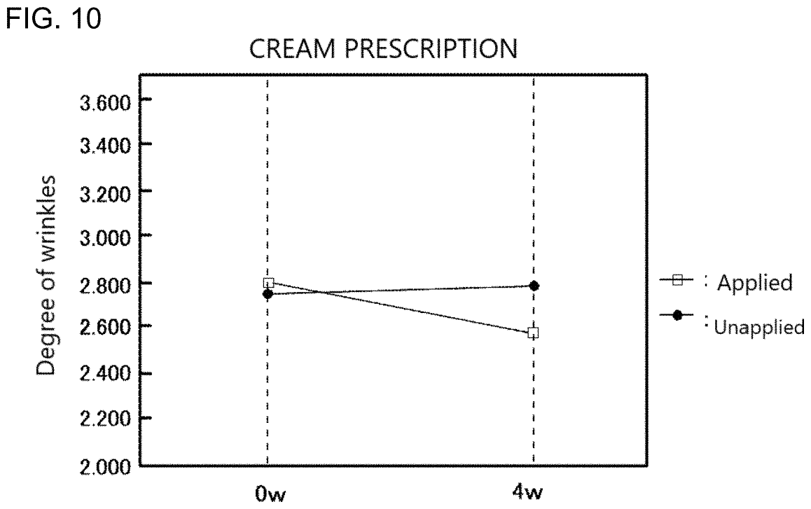

FIG. 9 is a graph which illustrates the measurement results of the water contents in the stratum corneum when the prescribed cream is applied.

At the applied dermal site, a significant rise in the water content in the stratum corneum was recognized after four weeks has passed (31.427±0.505) from the day 0 (30.136±0.157).

On the other hand, at the unapplied dermal site, there was no significant difference between the amount of the water content in the stratum corneum after four weeks has passed (30.200±0.190) from the day 0 (30.162±0.050).

Also, a significant increase was recognized in the amount of change in the cream applied dermal site (1.291±0.480), compared to that in the unapplied dermal site (0.038±0.190).

FIG. 10 is a graph which illustrates the measurement results of evaluation of the degree of wrinkles when the prescribed cream is applied.

At the cream applied dermal site, a significant decrease in the degree of wrinkles was recognized after four weeks (2.587±0.287) from the day 0 (2.800±0.254).

At the unapplied dermal site, a significant increase in the degree of wrinkles was recognized after four weeks (2.783±0.265) from the day 0 (2.750±0.231).

Also, a significant decrease was recognized in the amount of change at the cream applied dermal site (−0.217±0.088), compared to that in the unapplied dermal site (0.033±0.129) of four weeks later.

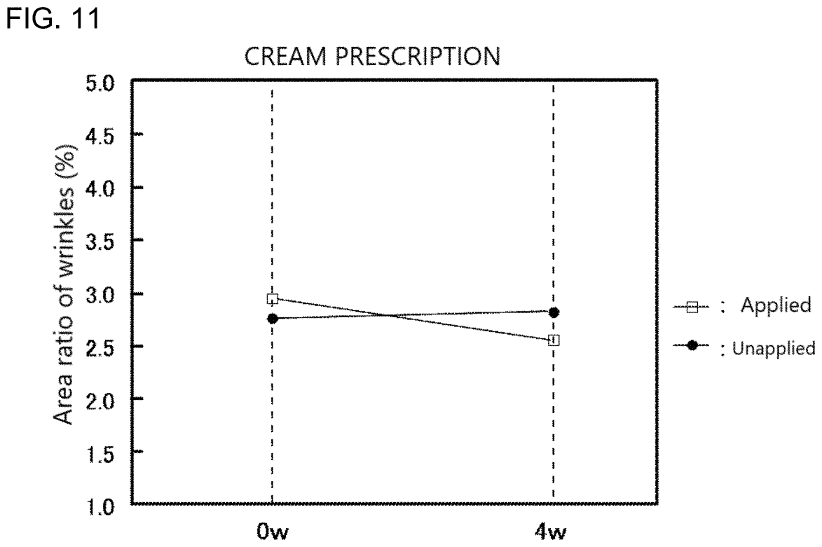

FIG. 11 is a graph which illustrates the measurement results of the area ratio of wrinkles when the prescribed cream is applied.

At the cream applied dermal site, a significant decrease in the area of wrinkles was recognized after four weeks (2.547±0.759) from the day 0 (2.959±1.003).

At the unapplied dermal site, no significant difference was recognized in the amount of change after four weeks (2.805±0.736) from the day 0 (2.767±0.741).

Also, a significant decrease was recognized in the amount of change at the cream applied dermal site (−0.412±0.734), compared to that in the unapplied dermal site (0.038±0.344).

(7) Test on Improvement in Fine Lines/wrinkles by the Prescribed Serum

A test was conducted on how well wrinkles of a human face caused by dryness would improve by the prescribed serum which contains diacylglycerol PEG.

The component composition of the prepared serum (wt %) is as follows:
WATER: 70.75
GLYCERIN: 10
SQUALANE: 8
BUTYLENE GLYCOL: 6
CETYL ALCOHOL: 4
PEG-12 GLYCERYL DIMYRISTATE: 2
PEG-23 GLYCERYL DISTEARATE: 2
GLYCERYL STEARATE: 2
PEG-45 STEARATE: 1.6
STEARYL ALCOHOL: 2
CHOLESTERYL MACADAMIATE: 1.5
SORBITAN STEARATE: 0.9
POLYGLYCERYL-10 STEARATE: 0.6
RETINYL PALMITATE: 0.55
ZEA MAYS (CORN) OIL: 0.45
ACRYLATES/C10-30 ALKYL ACRYLATE CROSSPOLYMER: 0.5
CHOLESTEROL: 0.1
POTASSIUM HYDROXIDE: 0.2
PHENOXYETHANOL: 0.3

(7-1) Test Method

The Water Content in the Stratum Corneum

The test was conducted under the following conditions:
Each of the subjects washed her face twice by a prescribed face cleansing cream and a prescribed facial wash, and leaving it (letting the face naturalized) for more than 20 minutes in the environmental test room (room temperature: 21±1° C., humidity: 50±5%).
The water content in the stratum corneum was measured on the part on which the serum was applied, and also on the part on which the serum was not applied on the day 0 and after four weeks as described below:
The test was conducted twice:
1st time: on the first day (day 0) before the subjects started to use the serum
2nd time: After the subjects used the serum twice a day (morning and night (after the bath)) every day for four weeks.
Measurement was made on the same parts (of the applied and unapplied parts) five times. The average value of the measurement results, from which the highest value and the lowest value were eliminated first, was adopted.

Area Ratio of Wrinkles

The test was conducted under the condition described as follows:
Each of the subjects washed her face twice by a prescribed face cleansing cream and a prescribed facial wash, and leaving it (letting the face naturalized) for more than 20 minutes in the environmental test room (room temperature: 21±1° C., humidity: 50±5%).
Replica was taken from the part on which the serum was applied, and also on the part on which the serum was not applied on the day 0 and after four weeks as described below:
The test was conducted twice:
1st time: on the first day (day 0) before the subjects started to use the serum.
2nd time: After the subjects used the serum twice a day (morning and night (after the bath)) every day for four weeks.

By using the image analysis system, the area ratio of wrinkles was measured from the replica taken.

Average Depth of the Total Amount of Wrinkles

The test was conducted under the condition described as follows:
Each of the subjects washed her face twice by a prescribed face cleansing cream and a prescribed facial wash, and leaving it (letting the face naturalized) for more than 20 minutes in the environmental test room (room temperature: 21±1° C., humidity: 50±5%).
Replica was taken from the part on which the serum was applied, and also on the part on which the serum was not applied on the day 0 and after four weeks as described below:
The test was conducted twice:
1st time: on the first day (day 0) before the subjects started to use the serum.
2nd time: After the subjects used the serum twice a day (morning and night (after the bath)) every day for four weeks.
By using the image analysis system, the average depth of total wrinkles was measured from the replica taken.

(7-2) Test Results

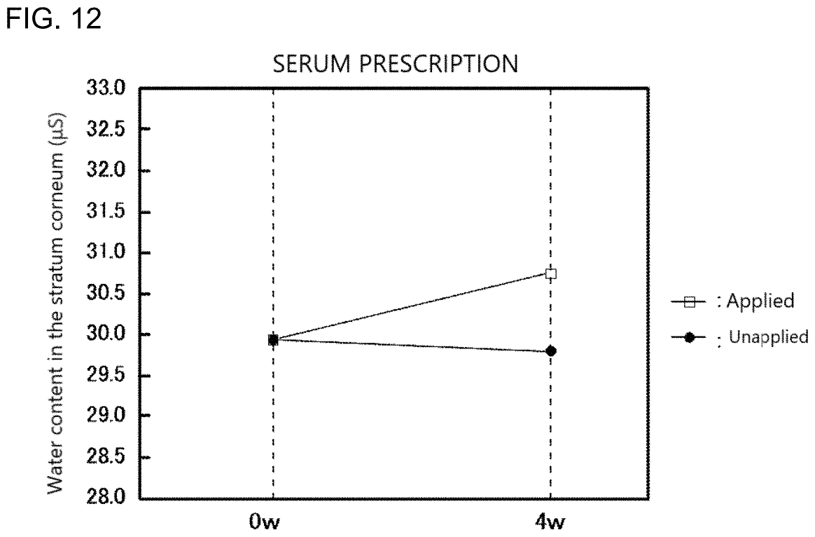

FIG. 12 is a graph which illustrates the measurement results of the water contents in the stratum corneum when the prescribed serum was applied.

At the applied dermal site, a significant rise was recognized from the day 0 (29.947±0.113) to that of four weeks later (30.753±0.256).

On the other hand, at the unapplied dermal site, there was no significant difference from the day 0 (29.960±0.210) to after four weeks (29.827±0.339).

Also, a significant increase was seen in the amount of change (0.807±0.246) on the serum applied dermal site, compared with the amount of change (0.133±0.366) on the unapplied dermal site.

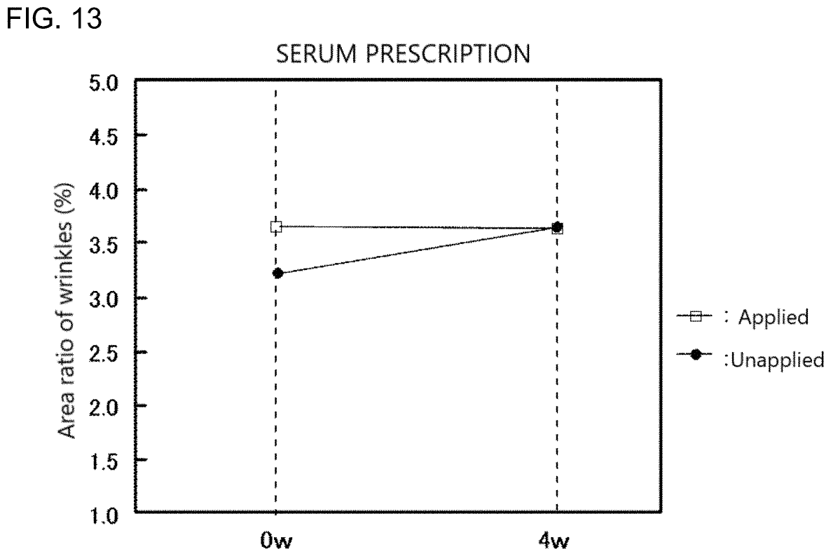

FIG. 13 is a graph which illustrates the measurement results of the area ratio of wrinkles when the prescribed serum is applied.

At the applied dermal site, no significant difference was recognized from the day 0 (3.649±1.196) to that after four weeks (3.621±±1.297) .

On the other hand, at the unapplied dermal site, a significant rise was recognized from the day 0 (3.229±1.004) till after four weeks (3.668±1.106).

Further, there was no significant difference between the applied dermal site and unapplied dermal site.

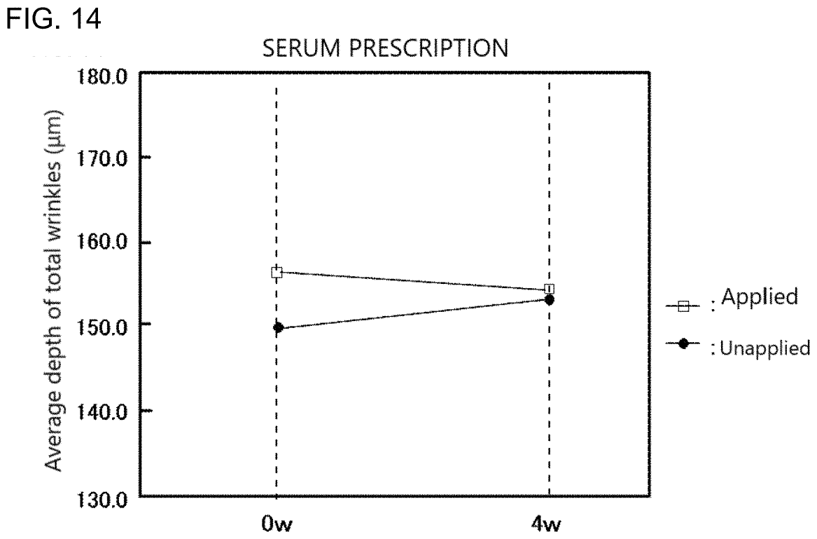

FIG. 14 is a graph which illustrates the measurement results of the average depth of total wrinkles when the prescribed serum is applied.

At the applied dermal site, no significant difference was recognized in the average depth of the total wrinkles from the day 0 (156.639±21.963) to that after four weeks (154.341±23.941).

On the other hand, at the unapplied dermal site, a significant rise was recognized from the day 0 (149.802±18.142) to that after four weeks (153.139±18.433).

Further, a significant decrease was seen in the amount of change (−2.028±6.826) on the serum applied dermal site, compared with the amount of change (3.337±4.904) on the unapplied dermal site.

Judging from the above-indicated test results of the three kinds of cosmetics, it has been confirmed that the reducing effect of wrinkles caused by dryness, i.e., the moisturizing effects of the cosmetics that contain diacylglycerol PEG adduct.

While the present invention has been described with reference to examples, the present invention is not limited to the examples and obvious modifications made thereby are also included in the present invention.

What is claimed is:

1. A method for enhancing expression of a moisturizing-related substance in an epidermis, comprising applying a diacylglycerol PEG adduct to a human epidermis as an active ingredient, wherein
the diacylglycerol PEG adduct is represented by 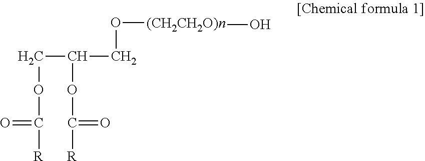
where number of carbons in R in a long-chain fatty acid is in a range of 11 to 23 and n in a polyethylene glycol chain is in a range of 11 to 46.

2. The method according to claim 1, wherein the diacylglycerol PEG adduct is selected from a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

3. The method according to claim 1, wherein the diacylglycerol PEG adduct permeates an epidermis in a solution state.

4. The method according to claim 1, wherein the diacylglycerol PEG adduct permeates an epidermis in a vesicle state.

5. The method according to claim 1, wherein the moisturizing-related substance is filaggrin.

6. The method according to claim 1, wherein the moisturizing-related substance is profilaggrin.

7. The method according to claim 1, wherein the moisturizing-related substance is caspase-14.

8. The method according to claim 1, wherein the diacylglycerol PEG adduct is one of ingredients of cosmetics or pharmaceuticals, and the method further comprises applying the cosmetics or pharmaceuticals on a surface of a human skin.

9. The method according to claim 2, wherein the diacylglycerol PEG adduct permeates an epidermis in a solution state.

10. The method according to claim 2, wherein the diacylglycerol PEG adduct permeates an epidermis in a vesicle state.

11. The method according to claim 2, wherein the moisturizing-related substance is filaggrin.

12. The method according to claim 2, wherein the moisturizing-related substance is profilaggrin.

13. The method according to claim 2, wherein the moisturizing-related substance is caspase-14.

14. The method according to claim 2, wherein the diacylglycerol PEG adduct is one of ingredients of cosmetics or pharmaceuticals, and the method further comprises applying the cosmetics or pharmaceuticals on a surface of a human skin.

15. The method according to claim 1, wherein the diacylglycerol PEG adduct is one of ingredients of a lotion, a cream or a serum, and the method further comprises applying the lotion, the cream or the serum onto a surface of human skin.

16. The method according to claim 2, wherein the diacylglycerol PEG adduct is one of ingredients of a lotion, a cream or a serum, and the method further comprises applying the lotion, the cream or the serum onto a surface of human skin.
